(19) United States
(12) Patent Application Publication
(10) Pub. No.: US 2017/0241872 A1
(43) Pub. Date: Aug. 24, 2017

(54) SYSTEM AND METHOD FOR INTRAOPERATIVE CELL STORAGE, PROCESSING, AND IMAGING

(71) Applicants: Aaron CHEUNG, Toronto (CA); Siu Wai Jacky MAK, Toronto (CA)

(72) Inventors: Aaron CHEUNG, Toronto (CA); Siu Wai Jacky MAK, Toronto (CA)

(21) Appl. No.: 15/507,055

(22) PCT Filed: Nov. 5, 2014
(86) PCT No.: PCT/IB2014/065829

§ 371 Date: Feb. 27, 2017

(30) Foreign Application Priority Data
Aug. 29, 2014 (IB) PCT/IB2014/064159

(51) Int. Cl. G01N 1/28; A61B 5/00; G01N 21/65; G01N 1/08; G01N 33/483; A61B 10/02; G01N 1/31

(52) U.S. Cl. CPC G01N 1/286; A61B 10/0266; A61B 5/0075; G01N 1/31; G01N 1/08; G01N 33/4833; G01N 21/65; G01N 2201/0826; G01N 2201/06113

(57) ABSTRACT

The present invention provides a system and method for collection, storage and processing of tissues and cells. The system includes a collection container with chambers for storing and processing tissues, which are controllably separated and maintain a physiologic environment for the tissues. The system also includes a fluidic device for isolating target cells of interest. The method includes receiving the tissue into a collection chamber, transferring the tissue to a processing chamber, dissociating the tis sue into single cells, and passing the single cells to a device for isolating one or more target cells.

### CROSS-REFERENCE TO RELATED APPLICATIONS

This application claims priority to PCT Application No. PCT/162014/064159, titled “MOLECULAR CELL IMAGING USING OPTICAL SPECTROSCOPY” and filed on Aug. 29, 2014, the entire contents of which are incorporated herein by reference.

### FIELD

The present disclosure relates to systems and methods for the storage and processing of surgical tissue samples into single cells and subsequent analysis using optical spectroscopy.

### BACKGROUND

Brain tumors are abnormal cell proliferations that occur in the central nervous system (CNS). It is estimated that there are over 23,000 new brain tumor cases in the United States resulting in over 14,000 deaths per year (Ostrom et al., 2013). Glioblastoma Multiforme (GBM), World Health Organization grade IV astrocytoma, is the most common and aggressive primary brain tumor in humans accounting for over 45% of all malignant brain tumors in the US (Ostrom et al., 2013). The current standard care for GBM involves a combination of chemotherapy with the oral methylating agent, temozolomide, radiation therapy, and/or maximal surgical resection. Although tumor shrinkage is observed following such treatments, brain tumor relapse is observed in around 90% of patients, resulting in a median survival of only 12 to 15 months (Stupp et al., 2009; Weller et al., 2013).

There is evidence that cancer is maintained and driven by stem-like cells known as cancer stem cells (CSCs), similar to organs where maintenance and homeostasis are driven by adult stem cells (Beck and Blanpain, 2013; Zhou et al., 2009). CSCs were first isolated from leukemia, and have since been isolated from many solid tumors including breast, colon, pancreatic, prostate, skin, head and neck, ovarian, lung and liver tumors (Al-Hajj et al., 2003; Bapat et al., 2005; Bonnet and Dick, 1997; Collins et al., 2005; Curley et al., 2009; Dalerba et al., 2007; Eramo et al., 2008; Fang et al., 2005; Kim et al., 2005; Lapidot et al., 1994; Monzani et al., 2007; O'Brien et al., 2007; Patrawala et al., 2006; Ponti et al., 2005; Quintana et al., 2008; Ricci-Vitiani et al., 2007; Schatton et al., 2008; Szotek et al., 2006; Vermeulen et al., 2008; Yang et al., 2008; Zhang et al., 2008). Brain tumor stem cells (BTSCs) were first isolated from post-operative brain tumor samples, including GBM, by sorting for surface markers that enriched for BTSCs in the CD133+ fraction (Singh et al., 2003; Singh et al., 2004). BTSCs exhibit properties of stem cells including their ability to self-renew in vitro as non-adherent neurospheres and multipotency in vitro, exhibited by the ability to differentiate into the three neural lineages including neurons, astrocytes, and oligodendrocytes. BTSCs also exhibit the same properties in vivo where the injection of as few as 100 CD133+ cells intracranially into immunodeficient xenograft models are able to reinitiate brain tumors that phenocopy the original patient, demonstrating multipotency of the BTSCs in vivo but more importantly, the ability of BTSCs to reinitiate the brain tumor. Finally, BTSCs exhibit self-renewal properties in vivo as CD133+ BTSCs can be isolated from the brain tumors of primary xenografts and serially transplanted into secondary xenografts and reinitiate brain tumor formation. These results demonstrate that a small population of cells within the brain tumor exhibit stem cell properties that allow these cells to initiate brain tumor formation. Note that the term CSCs may have different nomenclature in the field such as, but not limited to, tumor stem cells, tumor initiating cells, tumor progenitor cells, cancer initiating cells, or cancer progenitor cells. In this disclosure, the term CSCs encompasses all the cell types aforementioned. Similarly, this is also extended to BTSCs where the term brain tumor can be used as a prefix of the aforementioned terms to describe CSCs within brain tumors.

The existence of BTSCs may explain the high recurrence and mortality rates seen in brain tumor patients who have undergone standard care (Stupp et al., 2009; Weller et al., 2013). One of the characteristics of CSCs is that they are able to evade many standard care treatments. For example, BTSCs have been shown to exhibit resistance to common antineoplastic chemotherapeutic drugs (Chen et al., 2012; Eramo et al., 2006) and to radiation therapy via preferential upregulation of the DNA damage checkpoint response and increase in DNA repair capacity (Bao et al., 2006). This preferential chemo- and radiation-therapy resistance is not unique to CSCs of the brain but has also been shown for CSCs of breast, colon, ovarian, pancreas, and leukemia (Adikrisna et al., 2012; Alvero et al., 2009; Diehn et al., 2009; Dylla et al., 2008; Kreso et al., 2013; Li et al., 2008; Oravecz-Wilson et al., 2009; Tehranchi et al., 2010; Todaro et al., 2007).

Surgical procedures may not be able to target the removal of CSCs of the tumor apart from removing the bulk tumor itself. Therefore, the therapeutic resistance of CSCs is one mechanism by which tumor relapse may occur, as standard treatments are unable to target and remove CSCs, leaving them behind in patients. The residual CSCs are then able to reinitiate a tumor through their stem cell characteristics (self-renewal and multipotency) and lead to recurrence. Consequently, it would be beneficial to be able to distinguish CSCs from other tumor cells because eradication of the CSCs may be required to eliminate the cancer. In this context, the term distinguish refers to the ability to create contrast or identify one cell type, such as CSCs, from another, such as non-CSCs, bulk tumor cells, adult stem cells, or healthy tissue.

Optical spectroscopy may be used to identify target cells such as CSCs. The optical absorption and scattering properties of biological tissue depend on both the chemical and structural properties of the tissue and the wavelength of the interacting light. How these absorption and scattering properties of tissue change as a function of light can be particularly useful, as it is often unique to chemicals or structures in the tissue (the spectrum of the tissue). For example the absorption features of oxy- and deoxy-hemoglobin can be used to measure the oxygenation of blood and tissue, and the scatter changes caused by different cellular sizes can be used to detect precancerous and cancerous tissue. The field of measuring these changes in optical properties as a function of light is known as spectroscopy and the device to measure the light at the various wavelengths is known as a spectrometer. Spectroscopy has found a wealth of current and potential applications in medicine.

An example of optical spectroscopy is Raman spectroscopy, a rapid and nondestructive method to analyze the chemistry of a given material using light (Raman and Krishnan, 1928). Raman spectroscopy takes advantage of an optical property known as inelastic scattering that occurs when light interacts with matter. This inelastic scattering is unique to the molecular structures of the matter, thus providing a unique spectrum (or signature) of the matter that can be unambiguously distinguished and identified. Raman spectroscopy may provide neurosurgeons with an unambiguous and objective method to create contrast between tissues that are relevant to neurosurgery. For example, Raman spectroscopy may aid and assist neurosurgeons in distinguishing between healthy and tumor tissues, therefore minimizing the amount of tumour tissues left behind while preserving the critical healthy tissues, ultimately improving the surgical outcome of the patient. Studies using xenograft mouse models with transplanted brain tumor cells (Ji et al., 2013; Karabeber et al., 2014; Uckermann et al., 2014) and frozen human brain tumor sections (Kalkanis et al., 2014; Kast et al., 2014) have provided proof-of-principle of the potential of Raman spectroscopy in distinguishing healthy and tumor tissue. Raman measurements have been acquired from a number of different stem cell types ex vivo (Harkness et al., 2012; Hedegaard et al., 2010) but have not yet been acquired from BTSCs or from CSCs in vivo.

Surgical removal of brain tumors may be done using port-based surgery. Port-based surgery is a minimally invasive surgical technique where a port is introduced to access the surgical region of interest using surgical tools. Unlike other minimally invasive techniques, such as laparoscopic techniques, the port diameter is larger than the tool diameter. Hence, the tissue region of interest is visible through the port.

Tissue removal devices, such as the Myriad system (NICO Corp.), are commonly used to remove tissues from patients during port-based surgeries. Tissue removal devices typically store the removed tumor samples in a collection container connected to the tissue removal probe or surgical resector. In most cases, the stored tissue sample remains in the collection container until the end of the surgery before it gets processed at a remote laboratory. Therefore, there is a significant delay between the time the tissue sample is resected during surgery and when it gets processed. Furthermore, although the collection container is completely enclosed, the environment in the container does not mimic the in vivo environment. The delay in processing combined with the lack of proper in vivo storage can significantly impact the tissue sample's biology.

What is lacking in the field is a way to visualize and remove target cells such as CSCs intraoperatively, and to store and isolate target cells in a way that maintains their in vivo characteristics.

### SUMMARY

In this disclosure, a method and system is described to provide intraoperative storage of resected tissue samples that mimics in vivo conditions, enable efficient processing of tissue samples into single cells and distinguish target cells from non-target cells.

### BRIEF DESCRIPTION OF THE DRAWINGS

FIG. 1 is a flow chart that depicts the steps involved in port-based neurosurgery and the harvesting, storage, processing, probing, and sorting of the tissue into single cells in an intraoperative manner.

FIG. 2 is a schematic of a view down a port during neurosurgery.

FIG. 3 is a schematic that illustrates the methods and barriers involved in isolating BTSCs from brain tumors.

FIG. 4a is a schematic of the tissue container for the processing of tissue samples into single cells and the fluidic device for the probing and sorting of single cells.

FIG. 4b is a schematic of the mixing channel section of the fluidic device with the purpose to move cells from digestive enzymes in one channel to cell culture media in another channel.

FIG. 5 is a schematic illustrating the simultaneous use of a Raman microscope with a fluidic device.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

As used herein, the term in situ means in the tissue of origin; the term in vivo means within a living organism and refers to the location of tissues and/or cells in their native environment in the body. This in vivo location contains the environmental factors that are most ideal and/or suitable for the preservation of the tissue and/or cell biology; the term ex vivo means outside of a living organism; the term in vitro means within a culture dish, test tube or elsewhere outside a living organism; “target cells” means cells that are intended for identification or isolation, note that there could be multiple target cells simultaneously that are of interest for identification or isolation; “non-target cells” means cells that are not intended for identification or isolation; “surrounding tissue” means tissue outside of the tissue being measured; “adjacent cells” means cells within the same tissue as the cells being measured; “Raman Spectroscopy” includes fiber-based Raman systems incorporating transmissive grating or reflective grating, other variations of Raman spectroscopy including but not limited to Coherent anti-stokes Raman Spectroscopy (CARS), Shifted-excitation Raman difference spectroscopy (SERDS) and stimulated Raman Spectroscopy (SRS) and non-fiber based Raman systems; “Fluidic device” refers to any fluidic system, including a microfluidic system, that makes use of fabricated channels as a method to manipulate, control, transport fluid and/or cells through the use of passive capillary forces, or active forces, such as fluidic pumps, micropumps, or valves.

In this patent, the term distinguish refers to the ability to create contrast or identify one cell type, such as CSCs, from another, such as non-CSCs, bulk tumor cells, adult stem cells, or healthy cells.

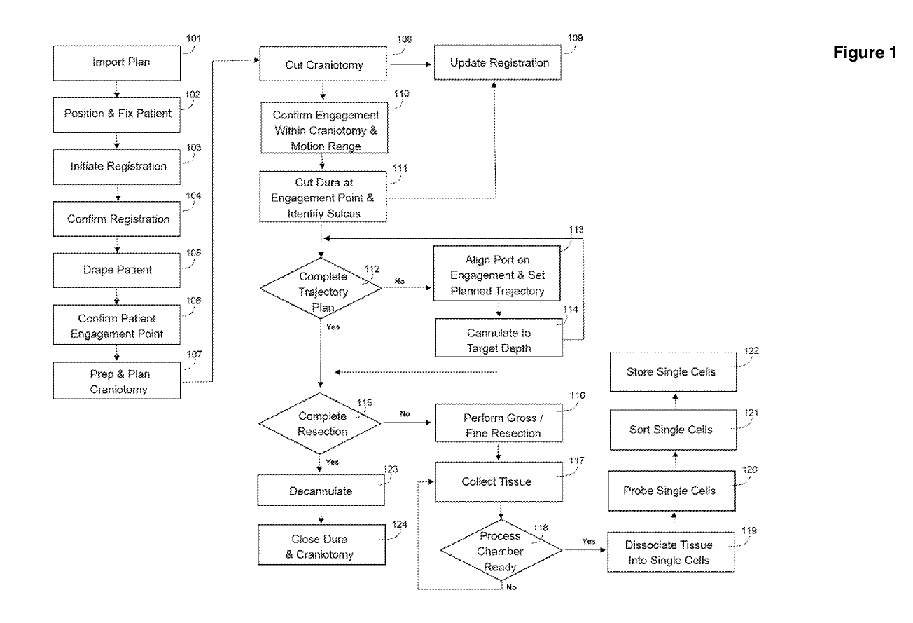

FIG. 1 is a flow chart illustrating the processing steps involved in a port-based surgical procedure. The example here describes identification of BTSCs but those skilled in the art will recognize that the method can be applied to other target cells, such as, but not limited to, other CSCs.

Surgical procedures are well known in the art. A first step involves importing a port-based surgical plan 101. An exemplary plan may include preoperative 3D imaging data (i.e., MRI, ultrasound, etc.), overlaying received inputs (i.e., sulci entry points, target locations, surgical outcome criteria, additional 3D image data information) on the preoperative 3D imaging data and displaying one or more trajectory paths based on the calculated score for a projected surgical path. An example of a process to create and select a surgical plan is outlined in the disclosure “PLANNING, NAVIGATION AND SIMULATION SYSTEMS AND METHODS FOR MINIMALLY INVASIVE THERAPY”, International Patent Application CA2014050272 which claims priority to U.S. Provisional Patent Application Ser. Nos. 61/800,155 and 61/924,993, which are hereby incorporated by reference in their entirety. The aforementioned surgical plan may be one example; other surgical plans and/or methods may also be envisioned.

Once the plan has been imported into the navigation system 101, the subject is affixed into position using a head or body holding mechanism. The head position is also confirmed with the subject plan using the navigation software 102.

The next step is to initiate registration of the subject 103. The phrase “registration” or “image registration” refers to the process of transforming different sets of data into one coordinate system. Data may be multiple photographs, data from different sensors, times, depths, or viewpoints. The process of “registration” is used in the present application for medical imaging in which images from different imaging modalities are co-registered. Registration is necessary in order to be able to compare or integrate the data obtained from these different modalities.

Those skilled in the art will appreciate that there are numerous registration techniques available and one or more of them may be used in the present application. Non-limiting examples include intensity-based methods which compare intensity patterns in images via correlation metrics, while feature-based methods find correspondence between image features such as points, lines, and contours. Image registration algorithms may also be classified according to the transformation models they use to relate the target image space to the reference image space. Another classification can be made between single-modality and multi-modality methods. Single-modality methods typically register images in the same modality acquired by the same scanner/sensor type, for example, a series of MR images can be co-registered, while multi-modality registration methods are used to register images acquired by different scanner/sensor types, for example in MRI and PET. In the present disclosure multi-modality registration methods are used in medical imaging of the head/brain as images of a subject are frequently obtained from different scanners. Examples include registration of brain CT/MRI images or PET/CT images for tumor localization, registration of contrast-enhanced CT images against non-contrast-enhanced CT images, and registration of ultrasound and CT.

Once registration is confirmed 104, the subject is draped 205. Typically draping involves covering the subject and surrounding areas with a sterile barrier to create and maintain a sterile field during the surgical procedure. The purpose of draping is to eliminate the passage of microorganisms (i.e., bacteria) between non-sterile and sterile areas.

Upon completion of draping 105, the next step is to confirm subject engagement points 106 and then prepare and plan craniotomy 107.

Upon completion of the preparation and planning of the craniotomy step 107, the craniotomy is carried out 108 in which a bone flap is temporarily removed from the skull to access the brain. Registration data is updated with the navigation system at this point 109.

The next step is to confirm the engagement within the craniotomy and the motion range 110. Once this data is confirmed, the procedure advances to the next step of cutting the dura at the engagement points and identifying the sulcus 111. Registration data is also updated with the navigation system at this point 109.

In an embodiment, by focusing the camera's gaze on the surgical area of interest, this registration update can be manipulated to ensure the best match for that region, while ignoring any non-uniform tissue deformation affecting areas outside of the surgical field. Additionally, by matching overlay representations of tissue with an actual view of the tissue of interest, the particular tissue representation can be matched to the video image to ensure registration of the tissue of interest. For example, the embodiment can:
Match video of post craniotomy brain (i.e. brain exposed) with imaged sulcal map;
Match video position of exposed vessels with image segmentation of vessels;
Match video position of lesion or tumor with image segmentation of tumor; and/or
Match video image from endoscopy up nasal cavity with bone rendering of bone surface on nasal cavity for endonasal alignment.

In other embodiments, multiple cameras may be used and overlaid with tracked instrument(s) views, and thus allow multiple views of the data and overlays to be presented at the same time, which may provide even greater confidence in a registration, or correction in more dimensions/views than provided by a single camera.

Thereafter, the cannulation process is initiated 112. Cannulation involves inserting a port into the brain, typically along a sulci path as identified in step 111, along a trajectory plan. Cannulation is an iterative process that involves repeating the steps of aligning the port on engagement and setting the planned trajectory 113 and then cannulating to the target depth 114 until the complete trajectory plan is executed 112.

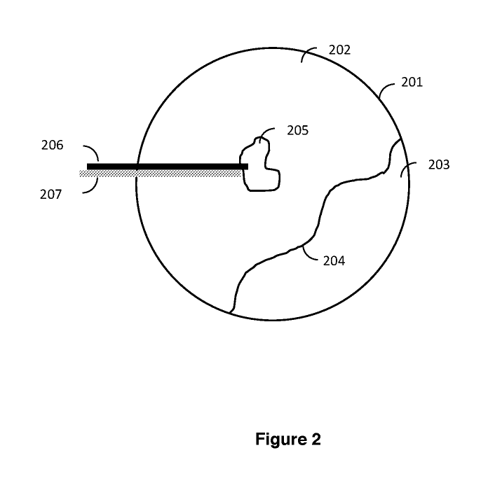

The surgeon then performs resection 115 to remove part of the brain and/or tumor of interest. Resection 115 is a continual loop including both fine and gross resection 116. During resection, the surgeon makes use of a resection tool within the port as described above and as further illustrated in FIG. 2. The port 201 is inserted during the cannulation process which provides the surgeon with a view of the tissue lying beneath. This tissue could represent both the tumor area 202 and/or the healthy area 203 separated by a tumor boundary 204. A portion of the tissue beneath the port may also represent the location of the BTSCs 205 which the surgeon does not know a priori. During resection, a surgeon typically will use a resector tool 206 that has two functions, the first of which is to suction the tissue within the tool, and the second of which is to resect the tissue within the tool. As an addition, this is described in PCT Application No. PCT/162014/064159 “MOLECULAR CELL IMAGING USING OPTICAL SPECTROSCOPY”, optical spectroscopy can be used in conjunction 207 with the resector tool allowing targeted isolation of target tissue or cells.

A problem that remains to be solved is a way to identify and isolate target cells, and in particular BTSCs intraoperatively. To solve this problem, four barriers to overcome are: i. identification of BTSCs, non-BTSC tumor cells, and healthy cells intraoperatively; ii. tissue resection in a minimally invasive manner in order to preserve the biology of the resected tissue and cells within; iii. storage of the tissues and cells within in a manner that mimics their in vivo environment to preserve their biology; and iv. isolation of target cells, such as BTSCs, from non-target cells, such as non-BTSC tumor cells, from the resected tissue.

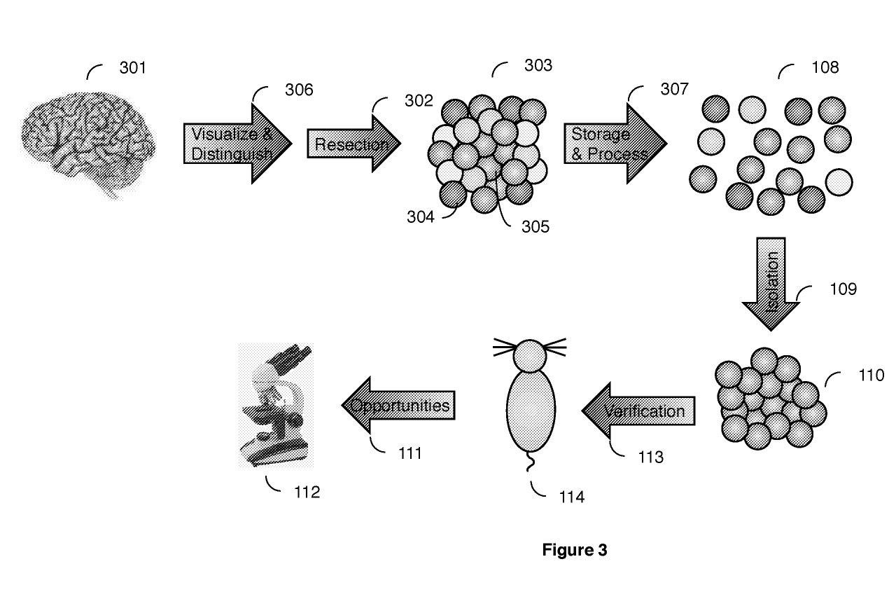

Regarding the first barrier, FIG. 3 illustrates the current methodologies used to surgically remove brain tumor samples, which are a source of BTSCs. The workflow begins with a subject, such as a patient, exhibiting a brain tumor 301 and to be treated using surgical resection 302 to remove the tumor. Resection 302 of brain tumor is largely done in a non-targeted fashion. Available tools to neurosurgeons for removing brain tumors include using preoperative images, such as MRI, which become increasingly inaccurate intraoperatively as the brain shifts in position relative to the skull during surgery. Neurosurgeons also commonly use color contrast to distinguish between healthy and tumor tissue, which is highly subjective. Ultimately, the removal of brain tumor is largely performed using non-targeted and non-quantitative methods. For this reason, the extracted brain tumor 303 is largely a heterogeneous population of cells that consists of both tumor mass cells 304 that make up the majority of the brain tumor and BTSCs 305 propagating the brain tumor. Note that it is likely that residual BTSCs are also left behind during neurosurgical removal of the brain tumor. Thus, the first barrier 306 relates to the limitations of current methods to visualize and distinguish target tissues or cells, such as BTSCs, intraoperatively. The first barrier of BTSC identification in situ is described in patent application PCT Application No. PCT/162014/064159 “MOLECULAR CELL IMAGING USING OPTICAL SPECTROSCOPY”.

Regarding the second barrier, once the target tissue or cell has been defined via imaging 306, the target tissue or cell is resected 302. The BTSC biology can be altered during procedures of brain tumor resection such as intraoperative manipulation, extraction technique and handling. Therefore, traditional devices such as ultrasonic aspirators and coagulation instruments that cause dissipation of thermal energy not only damage surrounding healthy brain tissue but may also compromise BTSCs' biology (McLaughlin et al., 2012). Hence, minimal manipulation of BTSCs intraoperatively and the use of nonablative instrumentation is preferred to preserve BTSC physiology. An example of a non-ablative instrument for tissue resection is the Myriad System (NICO Corp.). The Myriad system includes a resector tool which allows the isolation of tissue without crushing, or thermal and ablative damage on the sample, thereby preserving the tissue's biology (McLaughlin et al., 2012).

Regarding the third barrier, returning to FIG. 3, once a brain tumor sample 303 has been identified 306 in situ and resected 302, the tumor sample 303 is usually transported to a collection container connected to the tissue removal probe or surgical resector. In most cases, the stored tissue remains in the collection container until the end of the surgery before it goes through procedures for tissue storage and/or tissue processing 307. In the case of neurosurgery, the time between the start of tissue removal and the end of surgery may be in the order of hours, which is detrimental to the tissue's biology. Furthermore, although the collection container is completely enclosed, the environment in the container does not mimic the in vivo environment. For example, exposure to an atmosphere where the temperature, pH, oxygen conditions, and/or growth factors can vary from the tissue's native niche can alter the tissue's biology, genetics, epigenetics, chemistry, and metabolism instantaneously inducing BTSC death and differentiation (Bar et al., 2010; Soeda et al., 2009; Zhou et al., 2011). For these reasons, systems and methods that can store 307 tissues in a manner that preserves the tissue biology before the tissue is processed, followed by rapid processing 307 intraoperatively is a current unmet market need. In the case of brain tumors, the rapid processing and interrogation of brain tumor samples is important as the median survival for GBM patients is in the order of months. Rapid processing of a patient's brain tumor sample can provide important insights into, for example, their treatment regimen in a timely manner.

Returning to FIG. 1, a method for processing resected tissue is provided which overcomes the limitations of the current art. The resected tissue is collected 117 into a tissue container and stored under physiological conditions, as described in detail below. The tissue remains in the tissue container until the process chamber is ready 118. When the process chamber is ready 118, the tissue is moved to the process chamber, where the tissue is dissociated into single cells 119 using enzymatic and physical manipulation. Dissociated cells are then separated into cell clumps which are passed into a storage container and single cells which are passed into a fluidic system (described in detail below). Within the fluidic system, the cells are probed by optical spectroscopy 120. Based on spectral measurements, the cells are sorted 121 and stored 122.

Once resection is complete 115, the tissue is decannulated 123 by removing the port and any tracking instruments from the brain. Finally, the surgeon closes the dura and completes the craniotomy 124.

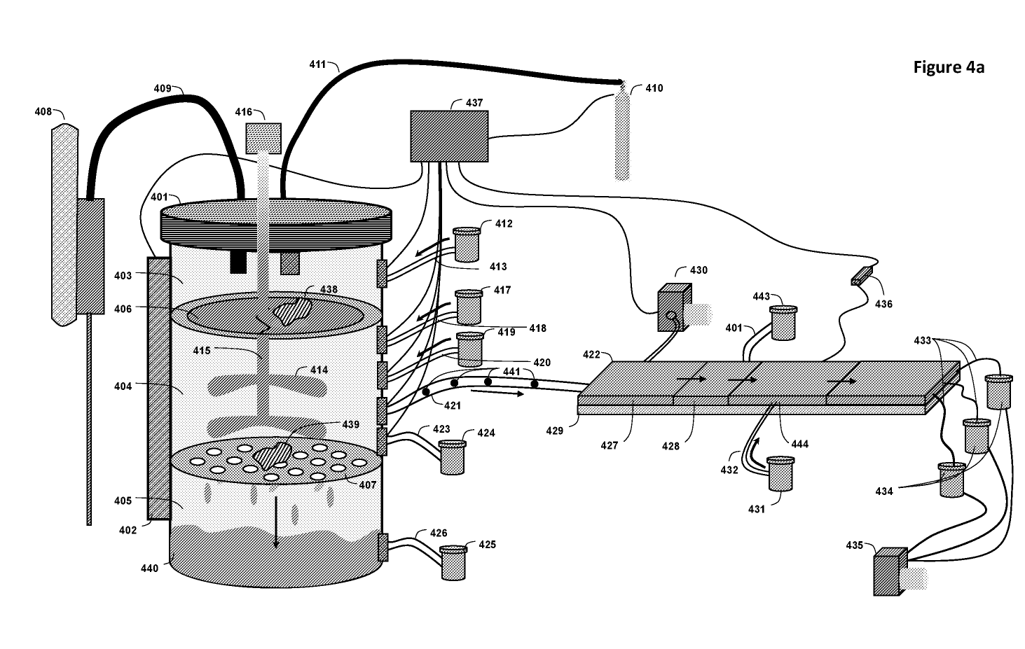

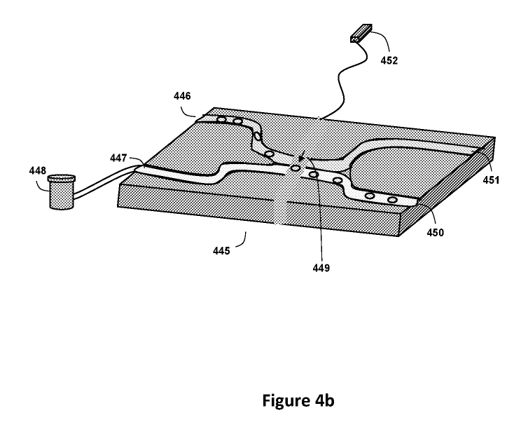

### DETAILED DESCRIPTION OF FIG. 4

As seen in FIG. 4a, the system and method for storing, processing and separating tissue includes a tissue processing container 401 which is provided with a temperature and humidity controller 402. The tissue processing container 401 includes a collection chamber 403, a process chamber 404 and a waste chamber 405. The collection chamber 403 is separated from the process chamber 404 by a controllable separator 406. The process chamber 404 is separated from the waste chamber 405 by a controllable solid filter 407.

The collection chamber 403 is connected to a resector tool 408 through a collection tube 409. The collection chamber 403 is also connected to a gas controller 410 through a gas inlet 411, and a media dispenser 412 through a media inlet 413.

The process chamber 404 is provided with rotatable blades 414 which are connected through a shaft 415 to a blade motor 416. The process chamber 404 is connected to a saline dispenser 417 through a saline inlet 418 and a digestive enzymes dispenser 419 through a digestive enzymes inlet 420. A cell outlet 421 leads from the process chamber 404 to a fluidic device 422 and a cell storage outlet 423 leads from the process chamber 404 to a first cell storage container 424.

The waste chamber 405 is connected to an excess fluid container 425 through an excess fluid outlet 426.

The fluidic device 422 includes a fluidic buffer 427 that converts a large fluidic channel to a small fluidic channel, a single cell filter 428 and a temperature control plate 429. The fluidic device 422 is connected to a first fluidic pump 430 and a media exchange reservoir 431 through a media inlet 432. Multiple channels 433 connect the fluidic device 422 to multiple cell storage containers 434, which are connected to a second fluidic pump 435.

The fluidic device is also connected to a laser 436 through a fiber bundle including excitation fibers and detection fibers.

The laser 436, gas controller 410, first fluidic pump 430, media dispenser 412, PBS dispenser 417, digestive enzyme dispenser 419, cell outlet 421, first cell storage outlet 423, temperature and humidity controller 402, and blade motor 416 are electronically connected to a control box 437.

During port-based surgery, the resector tool 408 is used to perform resection as described in FIG. 1 above. The resected tissue sample 438 is collected into the tissue processing container 401 via the collection tube 409. The tissue processing container 401 is an enclosed and sterile system. The environment of the tissue processing container 401 is customized by the temperature and humidity controller 402, and the gas controller 410 for nitrogen, oxygen, and carbon dioxide to control oxygen tension. The internal surface of the tissue processing container 401 may also be coated with (ECM), such as collagen, laminin, fibronectin or poly-L-ornithine, and have a 3D culture surface to further simulate in vivo conditions.

When the resected tissue sample 438 arrives in the tissue processing container 401, it is first collected in the collection chamber 403. The collection chamber 403 serves as an area where the resected tissue sample 438 is collected intraoperatively as surgery proceeds and stored before being processed. During this time, the biology of the tissue sample 438 can be preserved by modulating the variables (temperature, humidity, oxygen tension, ECM) mentioned previously to mimic the in vivo environment., For example, for BTSC, the ideal physiological temperature and humidity may be 37° C. and 95%, respectively, along with 5% CO₂ and 5%˜21% O₂. In addition, specific cell culture media 412 may be added into the collection chamber 403 which can further provide the tissue with favorable conditions to preserve its biology. For example, the use of favorable conditions that promote self-renewal of normal neural stem cells (NSCs) such as the use of growth factors including Fibroblast Growth Factor 2 (FGF2) and Epidermal Growth Factor (EGF), and ECM including laminin and Poly-L Ornithine, may help preserve BTSC biology.

In the collection chamber 403, the tissue samples are continually being collected and stored before processing. If the process chamber 404 is not ready for receiving the tissue sample 438, the controllable separator 406 remains in the closed position, preventing the tissue sample 438 from proceeding to the next stage. For example, the processing chamber 404 could be not ready because it is currently processing other tissue samples. This dual collection 403 and processing 404 chamber allows tissue samples to be collected and processed simultaneously. If there are no tissue samples being processed in the processing chamber 404, the process chamber 404 is ready to receive the tissue sample 438 from the collection chamber 403, and the controllable separator 406 opens, allowing the tissue sample 438 to drop to the processing chamber 404.

The role of the processing chamber 404 is to dissociate the tissue sample 439 into single cells. To achieve this, the controllable solid filter 407 opens to allow excess fluid 440 including, but not limited to, cell culture media, blood, and cerebral spinal fluid, to the waste chamber 405, while preventing solids, such as the tissue sample 439, from passing through. Once the processing chamber 404 is, devoid of liquids, the controllable solid filter 407 closes to prevent any more liquid from passing through. To prepare the tissue sample 439 for dissociation, a saline solution, such as Phosphate Buffer Saline (PBS) 417, is added to the processing chamber 404 to submerge the tissue sample 439. The blade 414 rotates to aid in the mixing and washing of the tissue sample 439 with PBS. After washing for 5 to 15 minutes, the blade 414 stops rotating, the controllable solid filter 407 opens to allow the used PBS to flow through, and then the controllable solid filter 407 closes again. This washing process can be repeated multiple times, such as up to three times, to ensure thorough washing of the tissue. Once the washing step is complete, the processing chamber 404 is emptied of excess fluids, and the controllable solid filter 407 is closed, then the tissue sample 439 is ready for dissociation. The tissue sample 439 is dissociated by the addition of digestive enzymes 419 such as, but not limited to, trypsin, collagenase, or accutase, into the processing chamber 404 sufficiently to submerge the tissue sample 439. Once the digestive enzyme is added, the blade 414 is turned on to aid in the mixing and dissociation of the tissue sample 439. The time required for digestive enzymes to dissociate tissue samples 439 into single cells varies with the digestive enzyme agent used and the size of the tumor sample. Generally, the process takes from anywhere between 15 minutes to an hour, but could also be beyond these time ranges. Note that the processing chamber 404 is also subjected to the same environmental controllable variables described for the collection chamber 403 described above (temperature, humidity, oxygen tension, ECM) to mimic the in vivo environment.

Once the dissociation step is complete, the single cells can be sent for further processing (described below). While the tissue sample 439 is being processed in the processing chamber 404, resected tissue samples 438 continue to be collected in the collection chamber 403. After the dissociation step is complete and the processing chamber 404 is devoid of tissues 439 or single cells, the next round of tissue samples 438 is deposited into the processing chamber 404 by opening the controllable separator 406 and the dissociation step is repeated. For these reasons, the tissue collection step and the tissue dissociation step can occur continuously and simultaneously throughout the surgical procedure without interruption.

The excess fluids 440 which include, but are not limited to, cell culture media, blood and cerebral spinal fluid, may be of significance for research purposes. For example, exosomes found in the serum of blood have significant roles in tumor pathogenesis (Abd Elmageed et al., 2014) and may serve as important diagnostic and prognostic factors. Therefore, it is advantageous to collect the excess fluid 440 into a container 425, which can then be used for downstream analysis (described below).

Returning to FIG. 3, once the brain tumor 303 has been stored and processed 307 into single cells 308, the fourth barrier relates to the ability to isolate 309 the BTSCs 310 from the non-BTSCs. In this context, non-BTSCs can include, healthy cells, non-BTSC tumor cells, and/or normal NSCs. Current state of the art to establish CSC lines 310 from tumor samples 303 include multiple methods of isolation 309 techniques. As an example, current methods for the isolation of BTSCs from brain tumors are provided here.

Isolation 309 of BTSCs 305 from a heterogeneous population of brain tumor 303 includes the use of cell surface markers such as CD133 for sorting through flow cytometry (Singh et al., 2004) and the use of favorable conditions that promote self-renewal of normal NSCs such as providing growth factors including FGF2 and EGF, ECM including laminin and Poly-L Ornithine (Pollard et al., 2009), and hypoxic oxygen concentrations such as 5% oxygen. The use of these techniques allows the isolation of BTSCs 305 from non-BTSCs 304 in the brain tumor sample 303.

Once BTSCs are isolated from the tumor cells, they are expanded. To expand newly isolated BTSCs 310, BTSCs can be propagated in vitro by several methods including 1) non-adherent or 2) adherent methods. In the non-adherent method, BTSCs are typically propagated in a low attachment container in the favorable conditions described above (growth factors and oxygen concentrations) promoting BTSCs to adhere to each other rather than the container and form spheres of cells known as neurospheres. These neurospheres can then propagate and expand in this configuration. In the adherent method, BTSCs are typically propagated in a container coated with a favorable ECM, promoting their attachment to the container. BTSCs will then be propagated in favorable conditions described above (growth factors and oxygen tension). These BTSCs, described as monolayers, can then propagate and expand in this configuration. Once BTSCs are stably propagating in vitro, they are considered a BTSC line 310.

The current need to culture BTSCs in vitro during isolation 309 is problematic as the culturing of BTSCs 310 in vitro can impose artifacts, such as, but not limited to, genetic and epigenetic changes, such that the BTSCs 310 do not resemble when they were in their in vivo state. The current norm of studying BTSCs that have been cultured in vitro may impact and confound any opportunities 311 such as research 312 to be performed on such BTSCs, yielding data that may not be relevant to their in vivo counterparts. There is currently a need to culture BTSCs in vitro because there are no methods to directly isolate the BTSCs in situ during surgery 302, the first barrier described above. This is described in PCT Application No. PCT/162014/064159 “MOLECULAR CELL IMAGING USING OPTICAL SPECTROSCOPY” the use of optical spectroscopy, such as Raman spectroscopy, to identify BTSCs in situ intraoperatively 306 during resection by comparing acquired spectra to a database of spectral signatures of known cell types. The use of optical spectroscopy to distinguish and isolate target cells, such as BTSCs, in situ can also be done after resection as a method to isolate 309 BTSCs 310 from a heterogeneous population of cells 308 intraoperatively. Note that it is also possible to utilize optical spectroscopy both during resection 306 and after processing 307 for the isolation 309 and confirmation of target cells 310.

Returning to FIG. 4a, after the tumor sample has been dissociated into single cells, the single cells flow from the cell outlet 421 into the fluidic device 422. Fluidic devices, such as continuous-flow fluidics, take advantage of a continuous liquid flow through fabricated channels. The liquid flow-through is driven by external pressure sources such as mechanical pumps, integrated mechanical micropumps, or a combination of capillary forces and electrokinetic mechanisms.

The dissociated single cells in the processing chamber 404 flow 441 into the fluidic device 422 along with the fluid from the processing chamber. The fluidic device may also include multiple channels for the cells to flow into and within the fluidic device to allow more efficient processing of the cells. The fluidic device is attached to a temperature control plate 429 to maintain the ideal in vivo temperature, such as 37° C., for the single cells while the cells are in the fluidic device 422. As the single cells flow into the fluidic device 422, the single cells will flow into a fluidic buffer 427 that converts a large fluidic channel to a small fluidic channel in which the single cells flow in a file of single cells. The cells then flow through a single cell filter 428 to ensure any residual cells that are clumped together are redirected through a cell outlet 442 and to a cell storage container 443 for later use and do not hinder the rest of the fluidic device 422.

When the single cells have passed through the single cell filter 428, they are still in digestive enzyme. Therefore, the cells will move into the mixing channel section 444 where the digestive enzyme will be removed and cell culture media will be added to preserve cell biology. FIG. 4b illustrates the mixing channel section 445 in detail within the fluidic device 422 where single cells in digestive enzyme 446 enter through a fluidic channel. In an adjacent fluidic channel, cell culture media 447, is input from the media exchange reservoir 431, 448. The goal of the mixing channel section 445 is to move 449 the single cells flowing through the fluidic device 422 from flowing in the channel with digestive enzyme 446 to the channel with cell culture media 450. The channel of digestive enzyme devoid of single cells 451 is then discarded. The movement of the single cells 449 may be performed using lasers from an external source 452 directed at a single cell to generate optical forces to push the single cell from the fluidic channel with digestive enzyme 446, 451 to the fluidic channel with cell culture media 447, 450.

The laser 452 generating optical forces to move 449 the single cells may also serve a dual purpose of interrogating the single cell. A preferred example where a laser can be used as both a cell sorter and identifier is Laser Tweezer Raman Spectroscopy (LTRS) (Chan et al., 2009; Chan et al., 2008). This technique combines the functionality of optical tweezers with that of confocal Raman spectroscopy into a single module, allowing the capture, identification, and sorting of cells to be done simultaneously with lasers. Optical tweezers enable a single cell to be captured in the focus area of the confocal Raman microscope which enables Raman acquisition on a single cell. After the Raman acquisition, the laser is directed on to the single cell along the plane of the fluidic device and optical forces from the laser move the single cell into a different channel. The laser may also be integrated into the fluidic device.

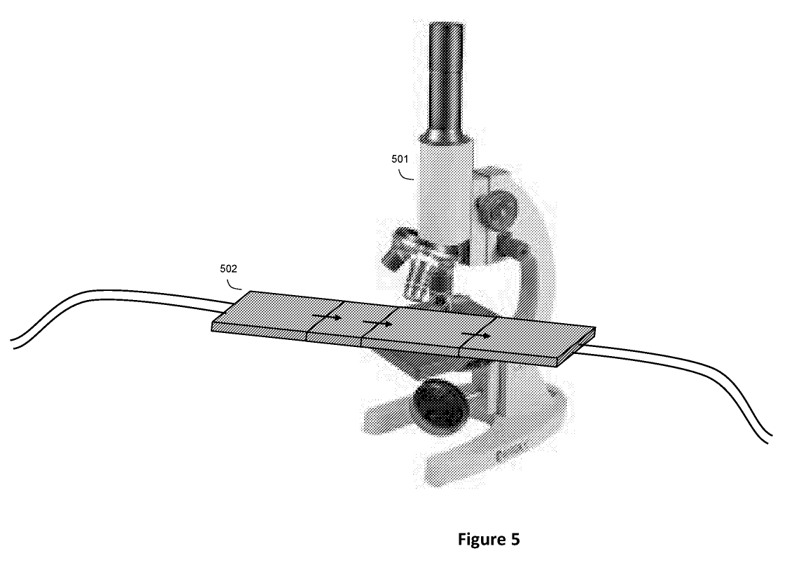

FIG. 5 illustrates a Raman microscope 501 integrated with the fluidic device 502 allowing interrogation of cells intraoperatively. Fluidic devices are on the orders of centimeters in length, which may be placed on a confocal microscope in the operation room away from the patient. Therefore, this enables studies and interrogation of cells intraoperatively with both optical and non-optical methods. It is important to note that the probing 120 of the cells and the sorting 121 need not occur simultaneously, as in LTRS, but may occur sequentially with multiple laser sources 436, 452. In this disclosure, probing 120 refers to the process of interrogating a cell's identity, such as, but not limited to, via optical spectroscopy, to determine whether it is a target cell of interest, for example, a BTSC, a non-BTSC tumor cell, healthy cell, or normal NSC. Sorting 121 refers to the process of separating the different target cell types after probing 120, for example, in to multiple fluidic channels and/or to discard non-target cells.

Returning to FIG. 4a, once the single cells in the cell culture media have been probed, for example, by Raman spectroscopy, the Raman spectra generated can be compared to a database of Raman spectra of known cell types. If a cell's spectrum is the same as that of a target cell spectrum within the database or is within a pre-determined range, then the cell is identified as a target cell. Based on the spectra comparison, the target cells of interest, such as BTSCs and non-BTSCs, are sorted into multiple fluidic channels 433 which flow into separate storage containers 434. Stored cells may be used for downstream applications, including, but not limited to, 1) direct implantation into immunodeficient mice; 2) long term storage in liquid nitrogen; 3) storage in containers 434 subject to the same environmental controllable variables described for the collection 403 and processing chamber 404 (temperature, humidity, oxygen tension, ECM); or 4) long term culture in the storage containers 434 similar to bioreactors by employing similar mechanics as the tissue processing container 401 described above. Finally, it is possible that not all the single cells from the processed tissue 439 will go through the fluidic device 422. Therefore it is possible to store any excess tissue 439 or cells in a storage container 424 connected to the process chamber 404 for future use.

In one example, fluids are moved through the fluidic device 422 by passive forces such as capillary forces. In another example the fluid may be moved through external forces such as active fluidics, for example fluidic pumps or micropumps 430, 435. The entire system as illustrated in FIG. 4a also involves multiple mechanics including the control of cell culture media 412, saline 417, digestive enzymes 419, cell, tissue, and fluid storages 424, 425, 434, 443, fluidic pumps 430, 435, lasers 436, gas 410, and temperature and humidity 402. A control box 437 may electrically control any or all of the multiple mechanics.

As mentioned previously, the excess fluids 440 may be of significance for study. Therefore, the fluidic device 422 may also be used to interrogate the excess fluids 440 by optical spectroscopy techniques, such as Raman Spectroscopy for important factors, such as exosomes and other extracellular vesicles within the blood or cerebral spinal fluid of patients.

### Verification of BTSCs

Returning to FIG. 3, prior to the use of BTSCs for research purposes, it is important to verify 313 their stem cell properties and confirm their identity as BTSCs. Similar to other stem cells, BTSCs should possess the two properties of stem cells, self-renewal and multipotency. Both these properties can be demonstrated in vitro where self-renewal is demonstrated via the routine propagation of the BTSCs as neurospheres or as a monolayer described above. Multipotency, or the ability to differentiate, can be demonstrated in vitro by placing BTSCs in differentiation inducing conditions by removing them from the self-renewal conditions described above (growth factors, oxygen tension, and substrate). For example, BTSCs can be placed in media lacking self-renewing factors FGF2 and EGF with the presence of serum to promote their differentiation into the three neural lineages, neurons, astrocytes, and oligodendrocytes, which can then be confirmed by molecular techniques including, but not limited to, immunocytochemistry and quantitative polymerase chain reaction. It is also vital to verify the stem cell properties of BTSCs in an animal or in vivo, where in vivo means within a living organism. Typically, to perform in vivo characterization, a xenograft assay is done, that is, BTSCs are injected intracranially into another species 314. The xenograft host is usually an immunocompromised rodent. Multipotency is demonstrated by the development of a brain tumor in the xenograft host by the injected BTSCs 310. The brain tumor in the xenograft should reflect pathologically the original brain tumor 301 in the subject, demonstrating the BTSCs' ability to differentiate into the different cell types comprising the original brain tumor. To demonstrate self-renewal in vivo, serial transplantation can be performed. This is demonstrated by isolating BTSCs from the brain tumor formed in the xenograft 310, re-transplanting into a secondary xenograft recipient, and showing that a brain tumor can form again in the secondary xenograft. In theory, this can be performed over multiple serial transplantations demonstrating the self-renewal of BTSCs in vivo. Upon verifying that BTSC lines have these stem cell properties, they are suitable for future use, including research 312, experimentation, and other opportunities 311.

### EXAMPLE 1

An example is provided here of the collection, storage and processing of BTSC, although it is equally applicable to the collection, storage and processing of other cells, such as other cancer stem cells or other cell types.

The collection, storage and processing is preferably carried out in the collection container described in FIGS. 4a and 4b. In operation, the collection container is controlled to be approximately 37° C. and the humidity is maintained at approximately 95% by the humidity controller.

Brain tumor tissue is resected using a resection tool, preferably as described in FIG. 2. The resected brain tumor tissue is collected through the collection tube to the upper collection chamber of the collection container and is stored there until the process chamber is empty and ready to receive tissue. Cell culture media that promote self-renewal of normal NSCs such as the use of growth factors including FGF2 and EGF, ECM including laminin and Poly-L Ornithine is added to the collection chamber from the cell culture media dispenser to cover the brain tumor tissue and a 5% CO₂ and 5% O₂ (hypoxic conditions) atmosphere is maintained by the gas controller.

When the process chamber is empty and ready to receive a sample, the separator partitioning the collection chamber from the process chamber opens to allow the tumor sample to pass to the process chamber. The solid filter partitioning the process chamber from the waste chamber opens to allow the cell culture media and other tissue-associated fluids and small solids to pass through to the waste chamber beneath the process chamber. The solid filter then closes and PBS is dispensed from the PBS dispenser into the process chamber and the tumor sample is washed in the PBS by mixing with the rotatable blade. The wash step with PBS is continued for 15 minutes, then the controllable filter is opened to allow the PBS to move to the waste chamber and the controllable filter is closed again. The PBS wash step is repeated two more times. After the tumor sample is washed, collagenase enzyme is dispensed from the digestive enzymes container. The rotatable blade then slowly rotates for 1 hour to dissociate the brain tumor tissue into single cells. The opening and closing of the separator and solid filter, the dispensing of solutions and the rotation of the blades is controlled by a control box.

After incubation in the enzyme solution and mixing with the rotatable blades to dissociate the tumor tissue into single cells, the cell outlet opens to allow passage of the cell suspension to the fluidic device. Cell movement to the fluidic device is maintained by a fluidic pump to bring the brain tumor cells into the fluidic device. The brain tumor cell suspension then continues to flow through the single cell filter, which partitions single cells from cell clumps. Cell clumps are diverted to a storage container. Single cells continue to move through the fluidic channel in the collagenase solution, until the channel parallels the cell culture containing channel.

A laser connected to the trypsin-containing channel through an excitation fiber transmits optical waves at 785 nm, which generates a physical force that moves the cell to the adjacent cell culture channel. At the same time, a detection fiber receives the transmitted optical spectrum from the probed cell and carries it to the control box, where it is compared with a consensus BTSC spectrum. If the spectra of the interrogated cell and a consensus BTSC match within 10% of values, a subsequent laser connected by an excitation fibre emits optical waves that elicit a force to direct the cell to a channel leading to a storage container for targeted cells. Other cells are identified by the optical spectroscopy as tumor cells or non-tumor cells and are directed to separate channels leading to storage containers, allowing sorting of multiple categories of cells. The BTSC are stored in the storage container under ideal physiological conditions, namely 5% CO₂, 5% O₂, 95% humidity, in NSC promoting cell culture media until the surgery is complete. Cells are then divided into an aliquot for storage in liquid nitrogen, aliquot for in vitro culture, and an aliquot for xenograft assay into a mouse xenograft model. The mouse xenograft model is used to test drug therapies for effectiveness at BTSC death.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A system for storing and processing one or more target cells comprising:
a. A collection container including a first chamber for receiving a tissue and a second chamber for processing the tissue into a plurality of cells, a media inlet for introducing a culture media to the collection container, a gas inlet for introducing gas from a gas controller to the collection container, an attached humidity and temperature controller for controlling the humidity and temperature of the collection container;
b. A controllable separator separating the first chamber from the second chamber;
c. A tissue inlet in the first chamber for receiving the tissue; and
d. A cell outlet in the second chamber connected to a fluidic device for separating one or more target cells from non-target cells.

2. (canceled)

3. The system as in claim 1, wherein an internal surface of the collection container is coated with extracellular matrix.

4. The system as in claim 1, wherein the collection container includes a third chamber for receiving excess fluids and a controllable filter separating the second chamber from the third chamber.

5. The system as in claim 1, wherein the tissue inlet in the first chamber is connected to a tissue resector tool through a tissue collection tube.

6. (canceled)

7. The system as in claim 1, further comprising one or more inlets in the second chamber for receiving a liquid saline solution and digestive enzymes.

8. The system as in claim 1, further comprising a movable means in the second chamber for dissociating the tissue.

9. (canceled)

10. (canceled)

11. (canceled)

12. (canceled)

13. The system as in claim 1, wherein a fluidic pump is connected to the fluidic device to propel the cells through the fluidic device.

14. The system as in claim 1, wherein the fluidic device comprises two or more channels for the cells.

15. The system as in claim 1, wherein the fluidic device comprises two or more channels for the cells, a temperature control plate, a single cell filter in at least one of the two or more channels, and one or more containers for receiving the target cells.

16. The system as in claim 1, wherein a laser is integrated in the fluidic device.

17. The system as in claim 14, wherein a laser is connected through a fiber bundle to at least one of the two or more channels of the fluidic device, the fiber bundle including excitation fibers to provide optical energy for cell movement between the two or more channels and to provide an optical spectra of the cells, and detection fibers to receive the emitted optical spectra.

18. A system for storing and processing one or more target cells comprising:
a. A collection container including a first chamber for receiving a tissue, a second chamber for dissociating the tissue into a plurality of cells, a third chamber for receiving an excess fluid from the tissue, an inlet for introducing gas from a gas controller to the collection container, an attached humidity and temperature controller for controlling the humidity and temperature of the collection container;
b. A controllable separator to separate the first chamber from the second chamber;
c. A controllable filter to separate the second chamber from the third chamber;
d. A tissue inlet in the first chamber connected to a tissue resector tool through a collection tube;
e. A media inlet in the first chamber for receiving a culture medium;
f. One or more inlets in the second chamber for receiving a liquid saline solution and digestive enzymes;
g. A movable means in the second chamber for dissociating the tissue;
h. A cell outlet in the second chamber for the cells;
i. An excess fluid outlet in the third chamber connected to a container for storing the excess fluid;
j. A fluidic device connected to the cell outlet in the second chamber, the fluidic device comprising two or more channels for the cells, a temperature control plate, a fluidic pump to propel the cells through the channels, a single cell filter in at least one of the channels;
k. a laser connected through a fiber bundle to at least one of the channels of the fluidic device, the fiber bundle including excitation fibers to provide optical energy for cell movement between the channels and to provide an optical spectra of the cells, and detection fibers to receive the emitted optical spectra, and one or more containers for receiving the target cells; and
l. At least one control box connected to the gas controller, the humidity and temperature controller, the culture medium inlet, the one or more second chamber inlet, the cell outlet, the excess fluid outlet, the laser and the fluidic pump.

19. A method for storing and processing one or more target cells comprising:
a. Receiving tissue into a collection chamber;
b. Maintaining the tissue in the collection chamber at a physiological temperature, humidity and atmosphere;
c. Passing the tissue from the collection chamber to a process chamber;
d. Dissociating the tissue into a plurality of single cells in the process chamber; and
e. Passing the single cells from the process chamber to a fluidic device for isolating one or more target cells.

20. The method as in claim 19, wherein the tissue is received intraoperatively from a tissue resector tool through a tissue collection tube.

21. (canceled)

22. The method as in claim 19, wherein a culture media comprising one or more of: serum; epidermal growth factor; fibroblast growth factor 2; and extracellular matrix, is added to the tissue in the collection chamber.

23. (canceled)

24. (canceled)

25. The method as in claim 19, wherein digestive enzymes are added to the process chamber to dissociate the tissue into a plurality of cells.

26. (canceled)

27. The method as in claim 19, wherein the tissue in the process chamber is mechanically stirred to dissociate the tissue into a plurality of single cells.

28. (canceled)

29. (canceled)

30. (canceled)

31. The method as in claim 19, wherein the one or more target cells are isolated in the fluidic device by:
a. Directing optical waves at the single cells within the fluidic device to move the single cells into a channel within the fluidic device; and
b. Passing the single cells from the channel to a storage container.

32. (canceled)

33. (canceled)

34. The method as in claim 31, wherein the optical waves are provided by a laser connected to the fluidic device through a fiber bundle including excitation fibers for providing excitation optical waves and detection fibers for measuring emitted optical spectra from the single cells, and the measured spectra are used to identify the one or more target cells.

35. The method as in claim 34, wherein the one or more target cells are identified by comparing the spectra of the single cells to a database of spectra to determine whether the spectra of the single cells matches the spectra of the one or more target cells.

36. (canceled)

37. (canceled)
